(19) United States
(12) Patent Application Publication
(10) Pub. No.: US 2011/0281928 A1
(43) Pub. Date: Nov. 17, 2011

(54) Process for the preparation of zofenopril and its pharmaceutically acceptable salts thereof

(75) Inventors: Mubeen Ahmed Khan, Maharastra (IN); Roop Singh Yadav, Maharastra (IN); Nikhil Rasiklal Trivedi, Maharastra (IN); Sachin Mahedo Lad, Maharastra (IN)

(73) Assignee: GLENMARK GENERICS LTD, MUMBAI, MAHARASTRA (IN)

(21) Appl. No.: 13/145,563

(22) PCT Filed: Jan. 24, 2010
(86) PCT No.: PCT/IN10/00034

§ 371 Date: Jul. 20, 2011

Related U.S. Application Data
(60) Provisional application No. 61/227,139, filed on Jul. 21, 2009.

(30) Foreign Application Priority Data
Jan. 23, 2009 (IN) 157/MUM/2009

(51) Int. Cl. A61K 31/40; A61P 9/12; A61P 9/10; C07D 207/16

(52) U.S. Cl. 514/423; 548/533

(57) ABSTRACT

The present invention relates to a process for the preparation of zofenopril and its pharmaceutically acceptable salts and a pharmaceutical composition thereof. The present invention also provides structurally novel compounds, which are useful intermediates in the synthesis of zofenopril.

### PRIORITY

This application is a 35 U.S.C. 371 National Stage Filing of International Application No. PCT/IN2010/000034, filed Jan. 1, 2010, which claims priority under 35 U.S.C. 119(a-d) to Indian Provisional IN 157/MUM/2009, filed on Jan. 23, 2009, and U.S. Provisional application 61/227,139, filed on Jul. 21, 2009 the contents of which, are incorporated by reference herein.

### BACKGROUND OF THE INVENTION

1. Technical Field

The present invention relates to a process for the preparation of zofenopril and its pharmaceutically acceptable salts and a pharmaceutical composition thereof. The present invention also provides structurally novel compounds that are useful intermediates in the synthesis of zofenopril.

2. Description of the Related Art

Zofenopril is an oral angiotensin-I converting enzyme (ACE) inhibitor which has been approved in EU for the treatment of mild to moderate essential hypertension and myocardial infarction. Zofenopril calcium is chemically described as (4S)-N-[3-(Benzoylsulfanyl)-2(S)-methylpropionyl]-4-(phenylsulfanyl)-L-proline calcium salt and has the following structure:

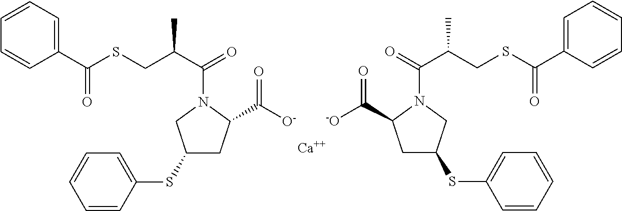

U.S. Pat. No. 4,316,906 (the '906 patent) describes mercaptoacyl derivatives and their pharmaceutically acceptable salts, which includes zofenopril, a pharmaceutical composition and method of treatment. The '906 patent discloses a process for the preparation of zofenopril calcium, which is illustrated by the scheme below:

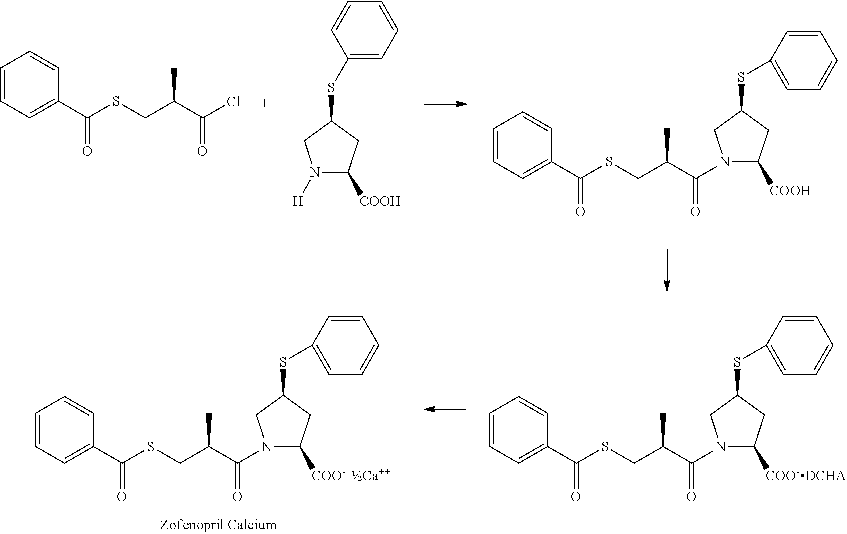

### SUMMARY OF THE INVENTION

The present invention relates to a process for the preparation of zofenopril and its pharmaceutically acceptable salts thereof.

In one aspect, the present invention provides a process for the preparation of zofenopril of formula I

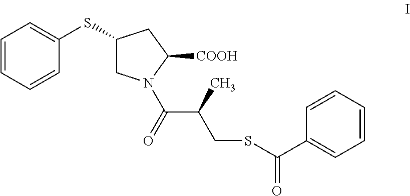

or a pharmaceutically acceptable salts thereof, comprising:
a) reacting protected cis-4-phenylthio-L-proline alkyl ester compound of formula IV or a salt thereof

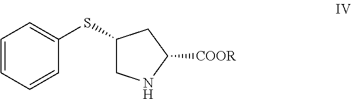

where R denotes hydrogen or linear or branched alkyl group or the benzyl group, with S-(−)-3-benzothio-2-methylpropionic acid compound of formula III or a salt thereof.

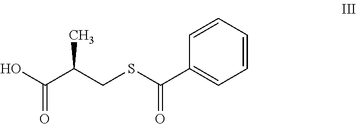

in the presence of coupling agents to form (4S)-1-[(2S)-3-(benzoylthio)-2-methylpropionyl]-4-(phenylthio)-L proline alkyl ester compound of formula II

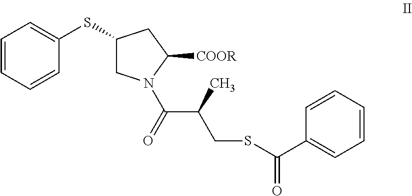

b) subjecting the resultant compound of formula II to deprotection to form the compound of formula I.

In a second aspect, the present invention provides a process for the preparation of protected cis-4-phenylthio-L-proline alkyl ester compound of formula IV or a salt thereof comprising reacting cis-4-phenylthio-L-proline compound of formula V or salt thereof

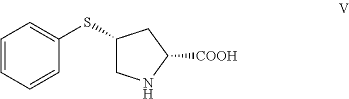

with an alcohol ROH, where R linear or branched alkyl group or the benzyl group.

In a third aspect, the present invention provides a process for the preparation of zofenopril of formula I or a pharmaceutically acceptable salts thereof, comprising:
a) reacting cis-4-phenylthio-L-proline benzyl ester compound of formula VI or a salt thereof

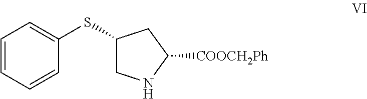

with S-(−)-3-benzothio-2-methyl propionic acid compound of formula III or ester derivative or a salt thereof

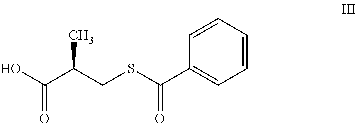

in the presence of coupling agents to form (4S)-1-[(2S)-3-(benzoylthio)-2-methylpropionyl]-4-(phenylthio)-L proline benzyl ester of structural formula VII,

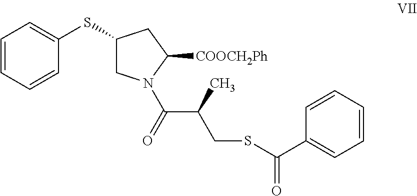

b) subjecting the resultant compound of formula VII to deprotection to form the compound of formula I.

In a fourth aspect, the present invention provides a process for the preparation of cis-4-phenylthio-L-proline benzyl ester compound of formula VI or a salt thereof

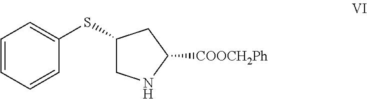

comprising reacting cis-4-phenylthio-L-proline compound of formula V or salt thereof

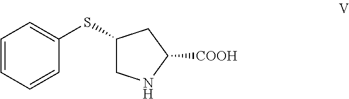

with benzyl alcohol.

In a fifth aspect, the present invention provides a compound (4S)-1-[(2S)-3-(benzoylthio)-2-methylpropionyl]-4-(phenylthio)-L proline benzyl ester of formula VII or a salt thereof.

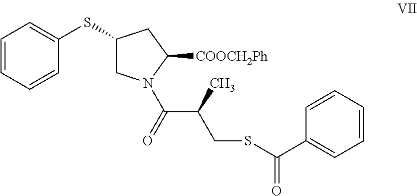

In a sixth aspect, the present invention provides a process for the preparation of zofenopril calcium comprising reacting zofenopril with a calcium derivative in the presence of an organic solvent.

In a seventh aspect, the present invention provides zofenopril calcium having impurity cis-phenyl thioproline acetamide of formula VIII

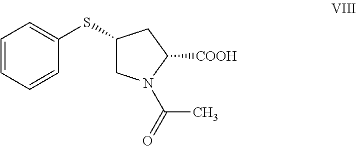

in an amount not more than about 1.0 area percent, as measured by HPLC.

In an eighth aspect, the present invention provides zofenopril calcium comprising cis-phenyl thioproline acetamide impurity of formula VIII in an amount not more than about 0.5% area percent, as measured by HPLC.

In a ninth aspect, the present invention provides zofenopril calcium comprising cis-phenyl thioproline acetamide impurity of formula VIII in an amount not more than about 0.1% area percent, as measured by HPLC.

In a tenth aspect, the present invention provides zofenopril in isolated solid form.

In another aspect, the present invention provides zofenopril in crystalline form.

In yet another aspect, the present invention provides zofenopril in amorphous form.

In yet further aspect, the present invention provides a process for preparing zofenopril, as previously described above, comprising the steps of:
a) providing a solution of zofenopril salt in a mixture of water and organic solvent.
b) subjecting the solution to hydrolysis;
c) recovering the desired solid form of zofenopril.

In a still yet another aspect, the present invention provides zofenopril free acid obtained by the process of present invention having an X-ray powder diffraction pattern characterized by with peak at about 9.45±0.2 degrees 2-theta, which is substantially in accordance with FIG. 2.

In another aspect, the present invention provides tertiary butyl amine salt of zofenopril which is preferably in crystalline form.

The present invention provides the zofenopril tertiary butyl amine salt characterized by X-ray powder diffraction pattern with characteristic peaks at about 3.0, 6.1, 8.4, 10.3, 11.2, 12.2, 12.3, 15.2, 16.5, 17.2, 18.2, 18.7, 18.9, 19.7, 20.6, 21.4, 23.5, 25.8 and 27.2±0.2 degrees 2-theta, which is substantially in accordance with FIG. 3.

In a still yet another aspect, the present invention provides zofenopril dicyclohexyl amine salt obtained by the process of the present invention having an X-ray powder diffraction pattern characterized by peak at about 6.9, 10.7, 11.8, 15.4, 17.0, 17.7, 18.2, 18.7, 19.1, 19.5, 21.3, 22.6 and 27.6±0.2 degrees 2-theta, which is substantially in accordance with FIG. 4.

A process for the preparation of zofenopril of formula I, in isolated solid amorphous or crystalline form

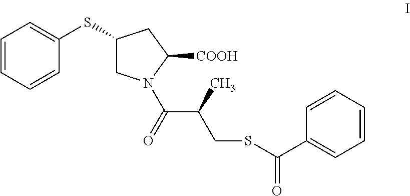

or a pharmaceutically acceptable salts thereof, comprising:
reacting cis-4-phenylthio-L-proline compound of formula V or a salt thereof

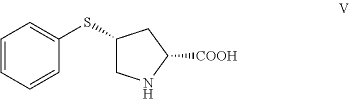

with S-(−)-3-benzothio-2-methyl propionic acid compound of formula III or ester derivative or a salt thereof

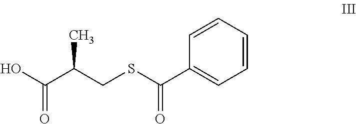

in the presence of coupling agent/s to give zofenopril of formula I.

In another aspect, the present invention provides zofenopril calcium having a mean particle size less than about 15 μm, wherein the zofenopril crystal calcium particles have a specific surface area from about 10 m²/g to about 15 m²/g, as measured by B.E.T. (Brunauer-Emmett-Teller) and floc shape as observed by SEM, which is substantially in accordance with FIG. 7.

In a yet further aspect, the present invention provides a pharmaceutical composition comprising zofenopril or its pharmaceutically acceptable salts and at least a pharmaceutically acceptable carrier.

### BRIEF DESCRIPTION OF THE DRAWINGS

FIG. 1: is a schematic representation of the process of the present invention.

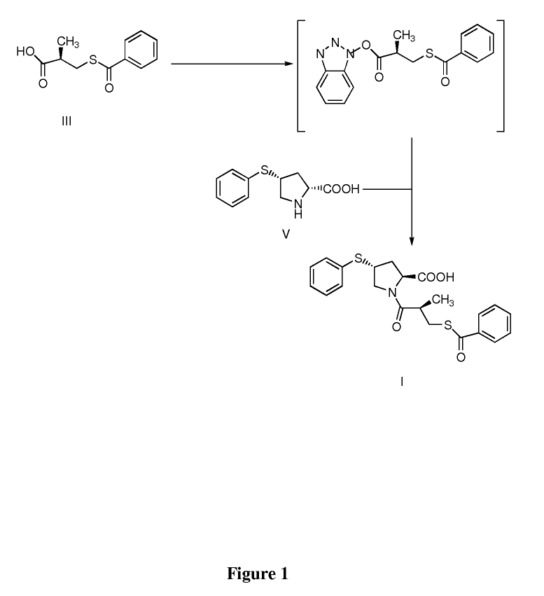

FIG. 2: is an X-ray powder diffraction pattern of zofenopril free acid prepared by Example 4.

FIG. 3: is an X-ray powder diffraction pattern of zofenopril tertiary butyl amine salt prepared by Example 1.

FIG. 4: is an X-ray powder diffraction pattern of zofenopril dicyclohexylamine salt prepared by Example 3.

FIG. 5: is an X-ray powder diffraction pattern of zofenopril calcium prepared by Example 5.

FIG. 6: is a differential scanning calorimetry thermogram curve of zofenopril calcium prepared by Example 5.

FIG. 7: Scanning Electron Microscope (SEM) photograph of zofenopril calcium crystal particles.

### DETAILED DESCRIPTION OF THE INVENTION

The present invention relates to a process for the preparation of zofenopril and its pharmaceutically acceptable salts thereof.

One of the impurities of zofenopril or its pharmaceutically acceptable salts is cis-phenyl thioproline acetamide of formula VIII,

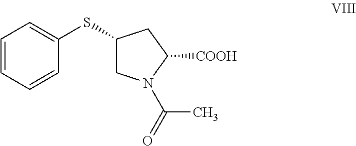

which is formed in coupling of intermediate compounds during the formation of the compound of formula I when ethyl acetate or acetic acid or its derivatives is used as solvents.

It would be advantageous to provide zofenopril in solid state and/or additional polymorphic forms. Whereupon, the availability of neutral zofenopril in solid form would be an added advantage in the preparation of pharmaceutically acceptable salts of zofenopril, like zofenopril calcium, which are useful in the preparation of pharmaceutical formulations for the treatment of hypertension.

The present invention provides a simple, ecofriendly, inexpensive, reproducible, robust processes for preparation zofenopril calcium and chemical compounds, which function as intermediates, in the process for the preparation therewith, which forthwith are viably adaptable on a commercial scale.

The present invention provides a process for the preparation of zofenopril of formula I

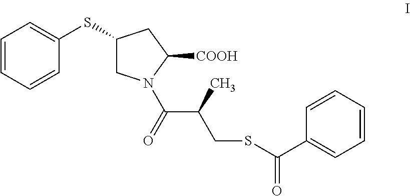

or a pharmaceutically acceptable salts thereof, comprising:
reacting protected cis-4-phenylthio-L-proline alkyl ester compound of formula IV or a salt thereof

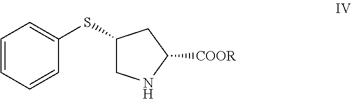

where R denotes a hydrogen or linear or branched alkyl group or the benzyl group, with S-(−)-3-benzothio-2-methyl propionic acid compound of formula III or a salt thereof

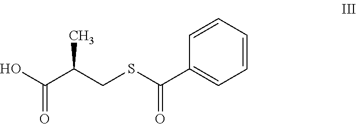

in the presence of coupling agent to form (4S)-1-[(2S)-3-(benzoylthio)-2-methylpropionyl]-4-(phenylthio)-L proline alkyl ester compound of formula II

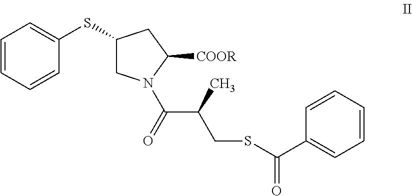

in which R has the same meaning as defined for formula IV
b) subjecting the resultant compound of formula II to deprotection to form the compound of formula I.

The reaction of (a) is carried out by a condensation reaction known in peptide synthesis, using suitable coupling agents. The coupling agents include, but are not limited to dicyclohexylcarbodiimide (DCC) and 1-hydroxybenzotriazole (HOBT) dicyclohexylcarbodiimide and 1-hydroxybenzotriazole 1-(3-dimethylaminopropyl)-3-ethylcarbodiimide hydrochloride, 1-(3-dimethylaminopropyl)-3-ethylcarbodiimide hydrochloride/1-hydroxybenzotriazole, 1-(3-dimethylaminopropyl)-3-ethylcarbodiimide hydrochloride/1-hydroxy-7-azabenzo-triazole, 1-(3-dimethylaminopropyl)-3-ethylcarbodiimide hydrochloride/N-hydroxysuccinimide, dicyclohexylcarbodiimide/1-hydroxy-7-azabenzotriazole, dicyclohexylcarbodiimide/N-hydroxysuccinimide, dicyclohexylcarbodiimide/3-hydroxy-3,4-dihydro-4-oxo-1,2,3-benzotriazine, dicyclohexylcarbodiimide/N-hydroxyphthalimide. Preferably, dicyclohexylcarbodiimide (DCC) and 1-hydroxybenzotriazole (HOBT).

Impurities in an API known in the art may arise from degradation of the API itself, which is related to the stability of the pure API during storage, and the manufacturing process, including the chemical synthesis. Process impurities include unreacted starting materials, chemical derivatives of impurities contained in starting materials, synthetic by-products, and degradation products. Impurities in zofenopril or any active pharmaceutical ingredient (API) are undesirable and, in extreme cases, might even be harmful to a patient being treated with a dosage form containing the API.

As noted above, one of the impurities which arises during the formation of zofenopril is cis-phenyl thioproline acetamide of formula VIII.

The International Conference on Harmonization (ICH) of Technical Requirements for Registration for Human Use Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process.

The reaction of (a) is normally and preferably effected in the presence of a solvent. There is no particular restriction on the nature of the solvent to be employed, provided that it has no adverse effect on the reaction or on the reagents involved and preferably it can dissolve the reagents to some extent. The solvents that can be used include but are not limited to halogenated solvents such as dichloromethane, ethylene dichloride, chloroform and the like or mixtures thereof; hydrocarbon solvents such as n-hexane, n-heptane, cyclohexane, toluene, xylene and the like or mixtures thereof; aprotic polar solvents such as N,N-dimethylformamide (DMF), dimethylsulfoxide (DMSO), dimethylacetamide (DMA), acetonitrile and the like or mixtures thereof.

The reaction can take place over a wide range of temperatures. In general, it would be convenient to perform the reaction at a temperature of from about −10° C. to about 120° C. or reflux temperatures of the solvents used. Preferably from about 30° C. to about 110° C., this may vary depending on the nature of the reactants and on the solvents employed.

The time required for the reaction to complete may also vary widely, depending on various factors, notably the reaction temperature and the nature of the reagents and solvent employed. However, provided that the reaction is effected under the preferred conditions outlined above, time required can be from about 1 hour to about 20 hours. Preferably from 1 hour to 10 hours.

The deprotection reaction of (b) is carried out by catalytic hydrogenation in the presence of hydrogen or by acid treatment. The hydrogenation catalysts that can be used for example platinum, palladium on charcoal carbon, platinum oxide, palladium dioxide, Raney nickel and the like; acids that can be used is selected from the group consisting of hydrochloric acid, sulfuric acid, trifluoroacetic acid, and the like.

The deprotection reaction is carried out in the presence of a solvent. The solvents that can be used include but are not limited to ketones such as acetone, ethyl methyl ketone, methyl isobutyl ketone and the like or mixtures thereof; esters such as ethylacetate, isopropylacetate, tertiary butyl acetate and the like or mixtures thereof; ethers such as tetrahydrofuran, 1,4-dioxane, and the like or mixtures thereof; hydrocarbon solvents such as n-hexane, n-heptane, cyclohexane, toluene, xylene and the like or mixtures thereof; aprotic polar solvents such as N,N-dimethylformamide (DMF), dimethylsulfoxide (DMSO), dimethylacetamide (DMA), acetonitrile and the like or mixtures thereof; or their aqueous mixtures.

The reaction can take place over a wide range of temperatures. In general, the reaction is carried at temperatures of about 30° C. to about 100° C. Preferably from about 30° C. to about 75° C., this may vary depending on the nature of the reactants and the solvents employed.

The time required for the reaction to complete may vary depending on the reaction temperature and the nature of the reagents and solvents employed. The time required for completion of the reaction can be from about 1 hour to about 20 hours. Preferably from about 1 hour to about 10 hours.

In another embodiment, the present invention, provides a process for the preparation of protected cis-4-phenylthio-L-proline alkyl ester compound of formula IV or a salt thereof

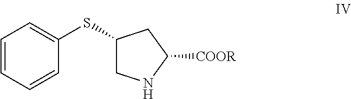

comprising reacting cis-4-phenylthio-L-proline compound of formula V or salt thereof

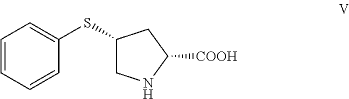

with an alcohol ROH, where R linear or branched alkyl group or the benzyl group.

The alkyl or aryl esters of compound of formula IV can be prepared by reaction of compound of formula V in the presence of organic solvent with an alcohol of general formula ROH where R is alkyl or aryl; preferably benzyl.

The alcohols that can be used include but are not limited to methanol, ethanol, isopropanol, tertiary butyl alcohol, benzyl alcohol and the like, preferably benzyl alcohol.

The organic solvents that can be used include but are not limited to ketones such as acetone, ethyl methyl ketone, methyl isobutyl ketone and the like or mixtures thereof; esters such as ethylacetate, isopropylacetate, tertiary butyl acetate and the like or mixtures thereof; ethers such as tetrahydrofuran, 1,4-dioxane, and the like or mixtures thereof; hydrocarbon solvents such as n-hexane, n-heptane, cyclohexane, toluene, xylene and the like or mixtures thereof; aprotic polar solvents such as N,N-dimethylformamide (DMF), Dimethylsulfoxide (DMSO), Dimethylacetamide (DMA), acetonitrile and the like or mixtures thereof; or their aqueous mixtures, preferably toluene.

The reaction can take place over a wide range of temperatures. In general, the reaction is carried at temperatures of about 30° C. to about 100° C. Preferably the reaction temperature can be from about 30° C. to about 75° C., and this may vary depending on the nature of the reactants and the solvents employed.

The time required for the reaction to complete may vary depending on the reaction temperature and the nature of the reagents and solvents employed. The time required for completion of the reaction can be from about 1 hour to about 10 hours, preferably from about 1 hour to about 5 hours.

The esters of compounds of formula IV and V, optionally, are converted into acid addition or carboxylic acid salts by reacting with hydrochloric acid, formic acid, oxalic acid, tartaric acid, methane sulfonic acid, benzene sulfonic acid, paratoluenesulfonic acid and the like, preferably paratoluenesulfonic acid.

The present invention provides a process for the preparation of zofenopril of formula I

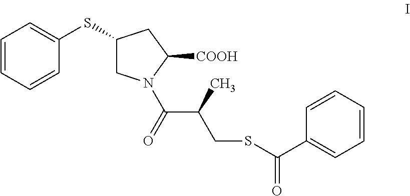

or a pharmaceutically acceptable salts thereof, comprising:
a) reacting cis-4-phenylthio-L-proline benzyl ester compound of formula VI or a salt thereof

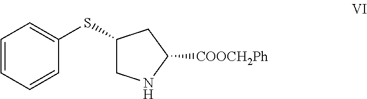

with S-(−)-3-benzothio-2-methyl propionic acid compound of formula III or ester derivative or a salt thereof

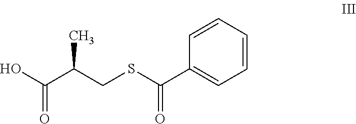

in the presence of coupling agents to form (4S)-1-[(2S)-3-(benzoylthio)-2-methylpropionyl]-4-(phenylthio)-L-proline benzyl ester of formula VII,

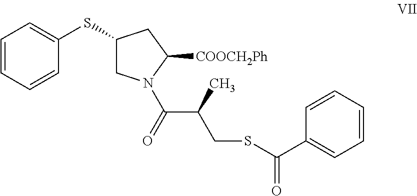

deprotecting the resultant compound of formula VII to form the compound of formula I.

The reaction of (a) is carried out by a condensation reaction known in peptide synthesis, using suitable coupling agents. The coupling agents include, but are not limited to dicyclohexylcarbodiimide and 1-hydroxybenzotriazole 1-(3-dimethylaminopropyl)-3-ethylcarbodiimide hydrochloride, 1-(3-dimethylaminopropyl)-3-ethylcarbodiimide hydrochloride/1-hydroxybenzotriazole, 1-(3-dimethylaminopropyl)-3-ethylcarbodiimide hydrochloride/1-hydroxy-7-azabenzo-triazole, 1-(3-dimethylaminopropyl)-3-ethylcarbodiimide hydrochloride/N-hydroxysuccinimide, dicyclohexylcarbodiimide/1-hydroxy-7-azabenzotriazole, dicyclohexylcarbodiimide/N-hydroxysuccinimide, dicyclohexylcarbodiimide/3-hydroxy-3,4-dihydro-4-oxo-1,2,3-benzotriazine, dicyclohexylcarbodiimide/N-hydroxyphthalimide preferably, dicyclohexylcarbodiimide and 1-hydroxybenzotriazole.

The reaction of (a) is normally and preferably effected in the presence of a solvent.

There is no particular restriction on the nature of the solvent to be employed, provided that it has no adverse effect on the reaction or on the reagents involved and preferably it can dissolve the reagents to some extent. The solvents that can be used include but are not limited to halogenated solvents such as dichloromethane, ethylene dichloride, chloroform and the like or mixtures thereof; hydrocarbon solvents such as n-hexane, n-heptane, cyclohexane, toluene, xylene and the like or mixtures thereof; aprotic polar solvents such as N,N-dimethylformamide (DMF), dimethylsulfoxide (DMSO), dimethylacetamide (DMA), acetonitrile and the like or mixtures thereof.

The reaction can take place over a wide range of temperatures. In general, it would be convenient to perform the reaction at a temperature of from about −10° C. to about 120° C. or reflux temperatures of the solvents used. Preferably the reaction temperature can be from about 30° C. to about 110° C., and this may vary depending on the nature of the reactants and on the solvents employed.

The time required for the reaction to complete may also vary widely, depending on various factors, notably the reaction temperature and the nature of the reagents and solvent employed. However, provided that the reaction is effected under the preferred conditions outlined above, time required can be from about 1 hour to about 20 hours, preferably from about 1 hour to about 10 hours.

Typically, the molar amount of compound of formula III may be about 1 to about 2 times the molar amount of the compound of formula VI, preferably about 1 molar amount; the molar amount of coupling agents may be about 1 to about 4 times the molar amount of the compound of formula IVa, preferably about 1 molar amount.

The deprotection reaction of step (b) is carried out by catalytic hydrogenation in the presence of hydrogen. The hydrogenation catalysts that can be used for example platinum, palladium on charcoal carbon, platinum oxide, palladium dioxide, Raney nickel and the like, preferably palladium-carbon.

The deprotection reaction is carried out in the presence of a solvent. The solvents that can be used, include but are not limited to, ketones such as acetone, ethyl methyl ketone, methyl isobutyl ketone and the like or mixtures thereof; esters such as ethylacetate, isopropylacetate, tertiary butyl acetate and the like or mixtures thereof; ethers such as tetrahydrofuran, 1,4-dioxane, and the like or mixtures thereof; hydrocarbon solvents such as n-hexane, n-heptane, cyclohexane, toluene, xylene and the like or mixtures thereof; aprotic polar solvents such as N,N-dimethylformamide (DMF), dimethylsulfoxide (DMSO), dimethylacetamide (DMA), acetonitrile and the like or mixtures thereof; or their aqueous mixtures.

The reaction can take place over a wide range of temperatures. In general, the reaction is carried at temperatures of about 30° C. to about 100° C., preferably from about 30° C. to about 75° C., which may vary depending on the nature of the reactants and the solvents employed.

The time required for the reaction to complete may vary depending on the reaction temperature and the nature of the reagents and solvents employed. The time required for completion of the reaction can be from about 1 hour to about 20 hours, preferably from 1 hour to about 10 hours.

The present invention provides a process for the preparation of cis-4-phenylthio-L-proline benzyl ester compound of formula VI or a salt thereof

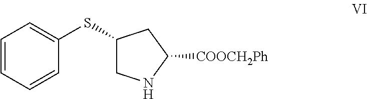

comprising reacting Cis-4-phenylthio-L-proline compound of formula V or a salt thereof

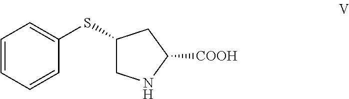

with benzyl alcohol.

The organic solvents that can be used include, but are not limited to, ketones such as acetone, ethyl methyl ketone, methyl isobutyl ketone and the like or mixtures thereof; esters such as ethylacetate, isopropylacetate, tertiary butyl acetate and the like or mixtures thereof; ethers such as tetrahydrofuran, 1,4-dioxane, and the like or mixtures thereof; hydrocarbon solvents such as n-hexane, n-heptane, cyclohexane, toluene, xylene and the like or mixtures thereof; aprotic polar solvents such as N,N-dimethylformamide (DMF), dimethylsulfoxide (DMSO), dimethylacetamide (DMA), acetonitrile and the like or mixtures thereof; or their aqueous mixtures, preferably toluene.

The reaction can take place over a wide range of temperatures. In general, the reaction is carried at temperatures of about 30° C. to about 100° C. Preferably the reaction temperature can be from about 30° C. to about 75° C., which may vary depending on the nature of the reactants and the solvents employed.

The time required for the reaction to complete may vary depending on the reaction temperature and the nature of the reagents and solvents employed. The time required for completion of the reaction can be from about 1 hour to about 10 hours, preferably from 1 hour to about 5 hours.

Similarly, as previously discussed, the esters of compounds of formula VI optionally can be converted into acid addition or carboxylic acid salts by reacting with hydrochloric acid, formic acid, oxalic acid, tartaric acid, methane sulfonic acid, benzene sulfonic acid, paratoluenesulfonic acid and the like, preferably paratoluenesulfonic acid.

The present invention provides (4S)-1-[(2S)-3-(benzoylthio)-2-methylpropionyl]-4-(phenylthio)-L proline benzyl ester compound of formula VII

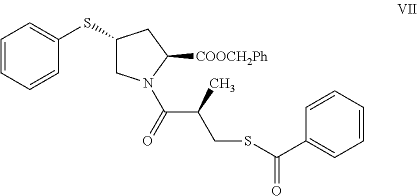

or a salt thereof.

Similarly, the present invention provides a process for the preparation of zofenopril of formula I, in isolated solid form,

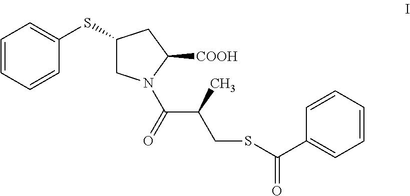

or a pharmaceutically acceptable salts thereof, comprising:
reacting cis-4-phenylthio-L-proline compound of formula V or a salt thereof

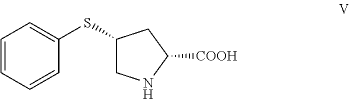

with S-(−)-3-benzothio-2-methyl propionic acid compound of formula III or ester derivative or a salt thereof

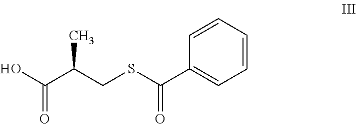

in the presence of coupling agent/s to give zofenopril of formula I.

The process conditions are as previously described above.

The present invention provides a process for the preparation of zofenopril calcium comprising reacting isolated solid zofenopril, prepared by the processes previously described, with a calcium derivative in the presence of an organic solvent.

The present invention provides zofenopril calcium having cis-phenyl thioprolineacetamide impurity of formula VIII

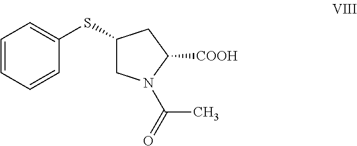

in an amount not more than 1.0 area percent, as measured by HPLC.

The present invention provides zofenopril calcium having cis-phenylthioproline acetamide impurity of structural formula VIII in an amount not more than 0.5% area percent, as measured by HPLC.

The present invention provides zofenopril calcium having cis-phenylthioprolineacetamide impurity of structural formula VIII in an amount not more than 0.1% area percent, as measured by HPLC.

Optionally, the compounds of formulae II and IV can be obtained by one pot reaction.

The compound of formula or a salt thereof used as one of the starting materials, can be prepared according to the method described in U.S. Pat. No. 4,559,178, which is incorporated herein by reference.

The compound of formula or a salt thereof used as one of the starting materials can be prepared according to the method described in U.S. Pat. No. 4,462,943, which is incorporated herein by reference.

After completion of the reaction, isolation of the desired compound from the reaction mixture can be carried out by common operation, but in consideration of the physical properties of the desired compound, crystallization, extraction, washing, column chromatography, etc. may be combined.

Optionally the present invention provides the process for the preparation of zofenopril calcium is carried out by one pot synthesis.

The compound of formula I is optionally purified by recrystallisation, using a solvent or mixture of solvents; or by converting into their corresponding pharmaceutically acceptable salts and then processed back to the compound of formula I.

There is no particular restriction on the nature of the pharmaceutical salts described above, provided that, where they are intended for therapeutic use, they are pharmaceutically acceptable. Examples include acid addition salts such as salts with mineral acids, especially hydrohalogenic acid (such as hydrofluoric acid, hydrobromic acid, hydroiodic acid or hydrochloric acid), nitric acid, carbonic acid, sulfuric acid or phosphoric acid; salts with lower alkylsulfonic acids, such as methanesulfonic acid, trifluoromethanesulfonic acid or ethanesulfonic acid; salts with arylsulfonic acids, such as benzenesulfonic acid or p-toluenesulfonic acid; and salts with organic carboxylic acids, such as acetic acid, propionic acid, butyric acid, fumaric acid, tartaric acid, oxalic acid, malonic acid, maleic acid, malic acid, succinic acid, benzoic acid, mandelic acid, ascorbic acid, lactic acid, gluconic acid or citric acid. These pharmaceutical salts are used as intermediates in the preparation and purification of compounds of formula I.

The intermediate salts of compound of formula I are converted into desired metal salts by reaction of the same with a derivative of metal alkali or alkaline earth metal such sodium, potassium, magnesium and calcium acid in the presence of a solvent, preferably a calcium salt.

Further processing option include the isolation of the free acid and/or the addition of seed crystals of said salt to obtain the desired crystals of said salt, i.e., calcium salt.

The solvent for salt dissolution that can be used is any liquid which has no adverse effect on the reaction and it can dissolve the reactants and the reagents to some extent. The solvents include ketonic solvents such as acetone, ethyl methyl ketone, methyl isobutyl ketone and the like and mixtures thereof or their aqueous mixtures; ester solvents such as ethyl acetate, n-propyl acetate, isopropyl acetate, tertiary butyl acetate and the like; nitriles such as acetonitrile, propionitrile, or mixtures thereof; preferably acetone, ethyl acetate, or acetonitrile.

The temperatures that can be used can range from about −20° C. to about 100° C., preferably from about 0° C. to about 70° C.

The time period for carrying out the reaction can be from about 5 minutes to about 10 hours, preferably 10 minutes to 5 hours. The precipitated salt of compound of formula I can be isolated from the reaction mixture by conventional methods known in the art, such as filtration or by evaporation of the solvent(s).

Like any synthetic compound, zofenopril or a pharmaceutically acceptable salt thereof can contain extraneous compounds or impurities that can come from many sources. These extraneous materials can be unreacted starting materials, by-products of the reaction, products of side reactions, or degradation products. Impurities in zofenopril or any active pharmaceutical ingredient (API) are undesirable and, in extreme cases, might even be harmful to a patient being treated with a dosage form containing the API. It is also known in the art that impurities in an API may arise from degradation of the API itself, which is related to the stability of the pure API during storage, and the manufacturing process, including the chemical synthesis. Process impurities include unreacted starting materials, chemical derivatives of impurities contained in starting materials, synthetic by-products, and degradation products. In addition to stability, which is a factor in the shelf life of the API, the purity of the API produced in the commercial manufacturing process is clearly a necessary condition for commercialization. Impurities introduced during commercial manufacturing processes must be limited to very small amounts, and are preferably substantially absent. For example, the International Conference on Harmonization of Technical Requirements for Registration for Human Use (ICH) Q7A guidance for API manufacturers requires that process impurities be maintained below set limits by specifying the quality of raw materials, controlling process parameters, such as temperature, pressure, time, and stoichiometric ratios, and including purification steps, such as crystallization, distillation, and liquid-liquid extraction, in the manufacturing process. The product mixture of a chemical reaction is rarely a single compound with sufficient purity to comply with pharmaceutical standards. Side products and byproducts of the reaction and adjunct reagents used in the reaction will, in most cases, also be present in the product mixture. At certain stages during processing of the API, zofenopril calcium, it must be analyzed for purity, typically, by HPLC, TLC or GC analysis, to determine if it is suitable for continued processing and, ultimately, for use in a pharmaceutical product. The API need not be absolutely pure, as absolute purity is a theoretical ideal that is typically unattainable. Rather, purity standards are set with the intention of ensuring that an API is as free of impurities as possible, and, thus, are as safe as possible for clinical use. As discussed above, in the United States, the Food and Drug Administration guidelines recommend that the amounts of some impurities be limited to less than 0.1 percent.

In one embodiment, zofenopril or a pharmaceutically acceptable salt thereof obtained by the above process comprises the following impurities namely L-proline impurity compound of formula (A), 4-hydroxyproline impurity compound of formula (B), N-(2-carbonyl pyrrolidine)-L-proline impurity, compound of formula (C), zofenopril thioester impurity compound of formula (D), and N,N′-dicyclohexylurea impurity compound of

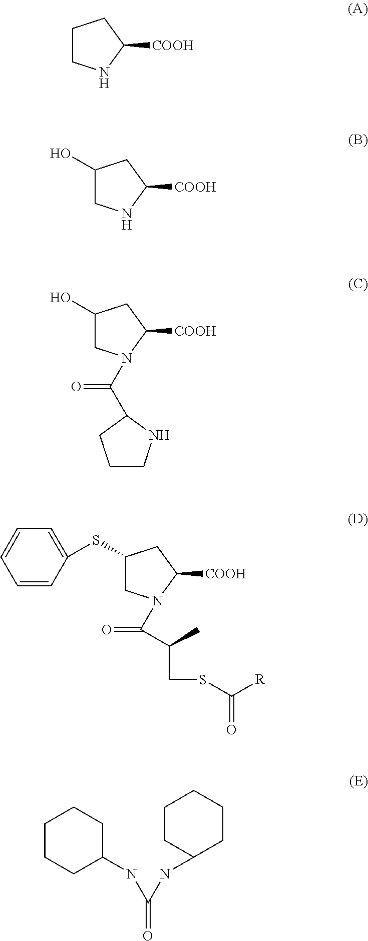

The total purity of the zofenopril or a pharmaceutically acceptable salt thereof obtained by the above processes is of at least about 98%, more preferably, at least about 99% and most preferably at least about 99.5%. The present invention provides a process for the preparation of zofenopril or a pharmaceutically acceptable salt thereof, wherein the level of purity is characterized in the manner described above, i.e., with the presence or lack thereof, of impurities

The present invention provides zofenopril in pure form, preferably isolated solid zofenopril. The zofenopril may be in crystalline form or in substantially amorphous form.

The '906 patent discloses the preparation of zofenopril as foamy solid using metal and alkyl amine salts of zofenopril as starting materials.

As the calcium salt of zofenopril is insoluble in organic solvents the purification of the final compound is difficult, so the isolation of zofenopril in isolated substantially solid form is essential; where the isolation is effectuated either by conventional crystallization techniques by direct reaction or by conversion into other pharmaceutically acceptable salts with purification via recrystallisation in solvents or mixture of solvents or acid base treatment and then converting directly into the desired calcium salt of zofenopril.

The difference in the physical properties of different solid state forms results from the orientation and intermolecular interactions of adjacent molecules or complexes in the bulk solid. Accordingly, polymorphs are distinct solids sharing the same molecular formula yet having distinct advantageous physical properties compared to other solid state forms of the same compound or complex.

The discovery of zofenopril in solid state provides a new opportunity to improve the performance of the corresponding active pharmaceutical ingredient. Zofenopril calcium or other salts, thereby can be produced using as starting materials, the solid forms of pure zofenopril having improved characteristics, such as stability, flowability, and solubility. Further, the solid state form of a compound may also affect its behavior on compaction and its storage stability.

The present invention provides a process for preparing zofenopril in isolated solid form comprising the steps of:
a) providing a solution of zofenopril salt in a mixture of water and organic solvent.
b) subjecting the solution to hydrolysis;
c) recovering the desired solid form of zofenopril.

In a) above, providing a solution of zofenopril salt in a mixture of water and organic solvent, the solution of zofenopril salt can be obtained by dissolving zofenopril salt in a mixture of water and suitable organic solvent(s). The solvent(s) that can be used in combination with water include, but are not limited to, alcohols, ketones, nitriles, aprotic polar solvents or mixtures thereof; halogenated solvents such as dichloromethane, chloroform, ethylene dichloride and mixtures thereof; alcohols include but are not limited to methanol, ethanol, n-propanol, isopropanol, n-butanol, isobutanol, and tertiary butyl alcohol and the like; ketonic solvents include but are not limited to acetone, methyl ethyl ketone, methyl isobutyl ketone, 2-butanone and the like; nitrile solvents include but are not limited to acetonitrile, propionitrile and the like; aprotic polar solvents may include N,N-dimethylformamide (DMF), dimethylsulfoxide (DMSO), N,N-dimethylacetamide (DMA) and the like; or mixtures thereof in various proportions without limitation.

The salt dissolution in (a) above is preferably carried out by dissolving a water soluble salt of zofenopril in a mixture of water and an organic solvent.

The salt of zofenopril used can be selected from salt forming ion compounds derived from such bases like metal ions, e.g., aluminum, alkali metal ions, such as sodium or potassium, alkaline earth metal ions such as calcium or magnesium, or an amine salt ion, of which a number are known for this purpose, for example, aralkylamines like, dibenzylamine, N,N-dibenzylethylenediamine, lower alkylamines like methylamine, tertiarybutylamine, procaine, hydroxy substituted lower alkylamines like tris (hydroxymethyl)aminoethane, lower alkyl-piperidines like N-ethylpiperidine, cycloalkylamines like cyclohexylamine or dicyclohexylamine, 1-adamantanamine, benzathine, or salts derived from basic amino acids like arginine, lysine or the like. Preferably, tertiarybutylamine. These and other salts which are not necessarily physiologically acceptable are useful in isolating or purifying a product acceptable for the purposes described. The salts are produced by reacting the acid form of the compound with an equivalent of the base supplying the desired basic ion in a medium in which the salt precipitates or in aqueous medium and then lyophilizing. The free acid form can be obtained from the salt by conventional neutralization techniques, e.g., with potassium bisulfate, hydrochloric acid, etc.

The temperature for getting clear and homogenous solution can range from about 25° C. to about 75° C. or boiling point of the solvents used, preferably from about 25° C. to about 40° C.

The solution obtained is optionally filtered through celite or diatomaceous earth to separate the extraneous matter present or formed in the solution by using conventional filtration techniques known in the art.

In (b) above, subjecting the solution to hydrolysis, the acids that can be used for hydrolysis include mineral acids such as hydrochloric acid, hydrobromic acid, preferably aqueous hydrochloric acid. The pH of the final solution may be, for example, from about 1 to about 5.

In c) above, recovering the desired solid form of zofenopril can be achieved by any conventional methods known in the art, for example, filtration.

The process may optionally include further drying of the product obtained from the solution by any method known in the art.

Crystallization may be induced by decreasing the solubility of zofenopril, e.g. by cooling the mixture, by evaporation of some of the solvents or by mixing with, e.g. by adding, some precipitating solvent or anti-solvent. The crystallization may start spontaneously, but it is likewise conducive to add seeds of the desired form of neutral zofenopril.

Crystallization is induced by mixing with, as for example addition of, a solution of an acid such that the pH of the final solution is still high enough to prevent significant degradation of the product. The organic solvent(s) is preferably a water miscible solvent(s) such as for instance, acetone, acetonitrile or a lower alkyl alcohol. The starting material of present invention is preferably a water soluble salt of zofenopril, for example a basic salt, particularly a tertiary butyl amine salt.

The starting material used in the salt dissolution process, described Herein may be a zofenopril salt, which may be of any polymorphic form known in the art. The resulting precipitate of zofenopril is generally in a solid form, which is either substantially in crystalline form or substantially in amorphous form. When the neutral zofenopril is crystallized, the crystals may be separated from the solution, e.g. by filtration or centrifugation, followed by washing with a washing liquid, preferably a solvent or a mixture in which the particular form of neutral zofenopril has a very low solubility, for example, an anti-solvent.

The substantially solid zofenopril can be dried under conditions which avoid degradation of the product, which can be from about 25° C. to about 35° C., and at reduced pressure of about 5 mbar to about 20 mbar, from about 1 hour to about 48 hours, preferably below about 25° C. and at reduced pressure of about 5 mbar for about 30 minutes to about 2 hours.

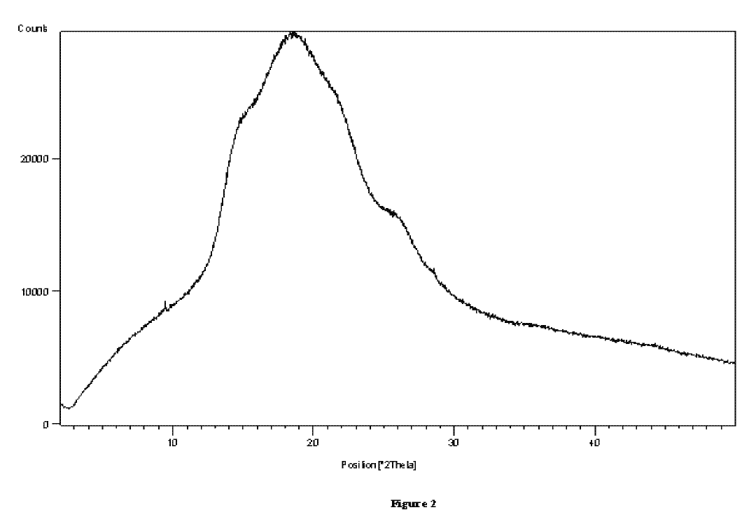

Zofenopril free acid obtained by the process of the present invention is characterized by X-ray powder diffraction pattern with peaks at about 9.45±0.2 degrees 2-theta, which is substantially in accordance with FIG. 2.

In yet another embodiment, the present invention provides tertiary butyl amine salt of zofenopril which is preferably in crystalline form.

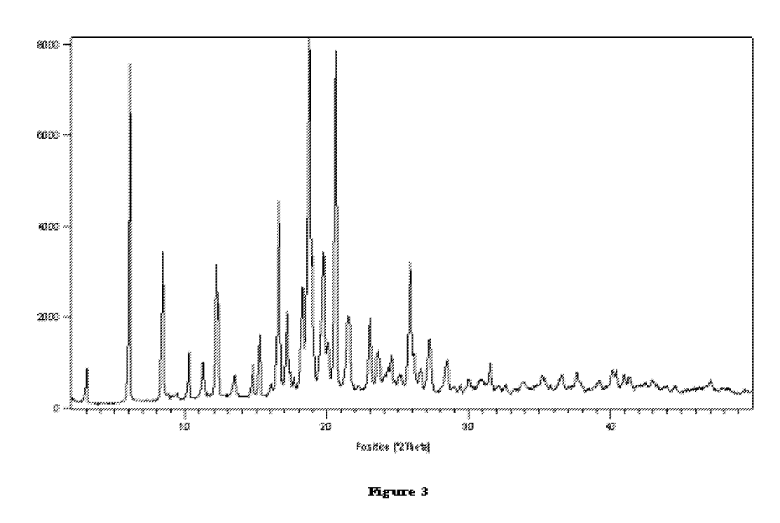

Zofenopril tertiary butyl amine salt of the present invention is characterized by X-ray powder diffraction pattern with characteristic peaks at about 3.0, 6.1, 8.4, 10.3, 11.2, 12.2, 12.3, 15.2, 16.5, 17.2, 18.2, 18.7, 18.9, 19.7, 20.6, 21.4, 23.5, 25.8 and 27.2±0.2 degrees 2-theta, which is substantially in accordance with FIG. 3.

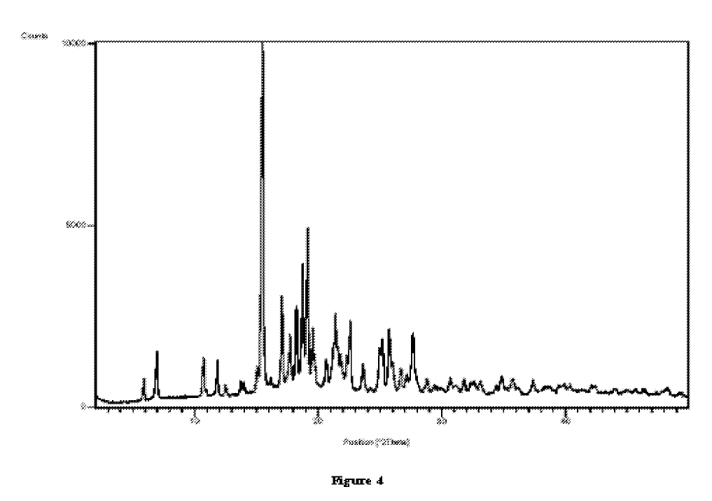

In still yet another aspect, the present invention provides zofenopril dicyclohexyl amine salt, obtained by the process of the present invention having an X-ray powder diffraction pattern with characteristic peaks at about 6.9, 10.7, 11.8, 15.4, 17.0, 17.7, 18.2, 18.7, 19.1, 19.5, 21.3, 22.6 and 27.6±0.2 degrees 2-theta, which is substantially in accordance with FIG. 4.

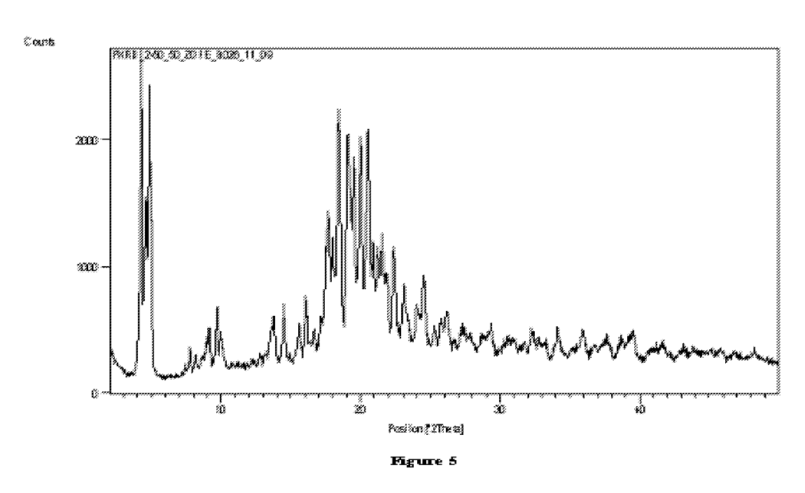

In one embodiment, zofenopril calcium obtained by the processes of present invention is characterized by an X-ray powder diffraction pattern with characteristic peaks at about 4.28, 4.58, 4.85, 4.99, 9.12, 9.7, 13.76, 14.47, 16.04, 17.48, 17.71, 18.01, 18.37, 18.53, 19.05, 19.52, 19.98, 20.57, 21.21, 21.93, 22.34 and 24.59±0.2 degrees two-theta, which is substantially in accordance with FIG. 5.

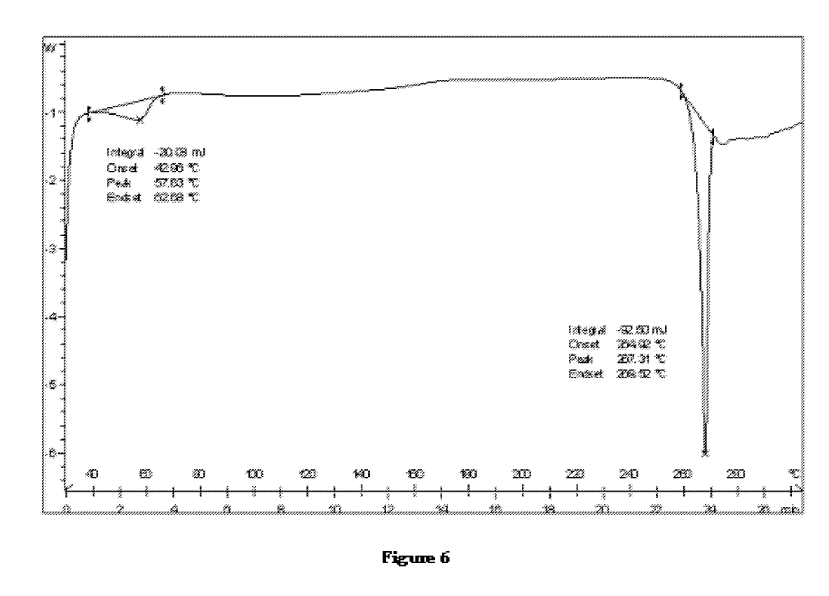

The zofenopril calcium obtained by the process of the present invention is further characterized by differential scanning calorimetry with an endotherm curve at about 267.31° C. with an onset at about 264.91° C. and an endset at about 269.52° C., which is substantially in accordance with FIG. 6.

X-ray powder diffraction measurements were performed on a Philips X'pert PRO Diffractometer using Cu Kα radiation (Cu Kα1=1.54060 \acute{Å}). The X-ray source is operated at 45 kV and 40 mA. Spectra are recorded at start angle from 2° to 50° 2θ, a step size 0.0167° with a time per step of 1000 seconds and DSC is measured by taking approximately 1-5 mg of sample was accurately weighed into an aluminum DSC pan with lid. The sample was placed then into a Mettler Toledo DSC822^{e} equipped with a liquid nitrogen cooling unit and allowed to equilibrate at 30° C. until stable heat flow response was seen. A dry nitrogen purge gas at a flow rate of 50 ml/min was used to produce the inert atmosphere and prevent oxidation of the sample during heating. The sample was scanned from 50-250° C. at rate of 10° C./min and resulting heat flow response was measured against temperature.

The zofenopril or a salt thereof according to the present invention has two asymmetric carbon atoms in the molecule; there are four stereoisomers possible having R and S configurations. The stereoisomers and a compound containing these in any proportion are both encompassed within the present invention. The stereoisomers, for example, can be synthesized by using optically resolved raw material compounds or can be obtained by subjecting synthesized zofenopril or a salt thereof to optical resolution, if desired, using a conventional optical resolution or separation method.

The zofenopril or a salt thereof according to the present invention may be allowed to stand in the air or recrystallized to absorb water, thereby having adsorbed water or may become a hydrate. The water-containing compounds are encompassed within the present invention. In addition, solvates thereof each containing any amount of a solvent are also encompassed within the present invention.

In yet another embodiment, zofenopril or its pharmaceutically acceptable salts like zofenopril calcium obtained by the processes described above has residual organic solvents or organic volatile impurities comprises less than the amount recommended for pharmaceutical products, as set forth for example in ICH guidelines and U.S. pharmacopoeia; less than about 600 ppm of dichloromethane, less than 3000 ppm of methanol, ethanol, ethyl acetate, isopropyl alcohol, less than about 100 ppm of acetonitrile and toluene.

The different physicochemical properties of the active ingredient and those of its excipients are to be considered, as these properties affect the process and formulation properties of the compound. Various important physicochemical properties include but are not limited to particle sizes, density (bulk density and tapped density), compressibility index, Hausner's ratio, angle of repose, etc. Particle sizes of active pharmaceutical ingredient can affect the solid dosage form in numerous ways. For example, content uniformity (CU) of pharmaceutical dosage units can be affected by particle size and size distribution. This will be even more critical for low-dose drugs and satisfactory dosage units of low doses cannot be manufactured from a drug that does not meet certain particle size and size distribution specifications. Also particle sizes play an important role in dissolution of active ingredient form the final dosage form for certain drugs like zofenopril because of their poor solubility.

Hence, these physicochemical properties not only affect the processes of the preparing the pharmaceutical formulations but also affect the performance of the pharmaceutical product both in vitro and in vivo.

The D10, D50, and D90 values are useful ways for indicating a particle size distribution. D90 is a size value where at least 90 percent of the particles have a size smaller than the stated value. Likewise D10 refers to 10 percent of the particles having a size smaller than the stated value. D50 refers to at least 50 percent of the particles having a size smaller than the stated value and D [4,3] value refers to a mean particle size. Methods for determining D10, D50, D90 and D [4,3] include those using laser light diffraction with equipment sold by Malvern Instruments ltd.

In the field of pharmaceutical formulation, it is notable that particle size plays a pivotal role in the solubility properties of an API, like zofenopril calcium. Particle size reduction techniques are employed to increase a compound's solubility. Particle size reduction increases the surface area of the solid phase that is in contact with the liquid medium. However, particle size reduction cannot alter the solubility of the compound in a solvent, which is a thermodynamic quantity. At instances where the rate of dissolution of a poorly soluble drug is the rate limiting factor in its rate of absorption by the body, it is recognized that the bioavailability of such drugs may be enhanced when administration occurs in a finely divided state. Further, particle size can also affect how free crystals or a powdered form of a drug will flow past each other, which in turn, has consequences in the production process of pharmaceutical products containing the drug.

The specific surface area of an active pharmaceutical ingredient may be affected by various factors. It is recognized that there is an inverse relationship between surface area and particle size; where the smaller the particle size, the higher the surface area. Whereupon, the available surface area for drug dissolution correlates to the rate of dissolution and solubility. A greater surface area enhances both the solubility and the rate of dissolution of a drug, which in turn, may improve its bioavailability and potentially its toxicity profiles.

The lack of solubility of a drug poses a challenge. Solubility may affect the bioavailability of a poorly water soluble active ingredient.

Hence, there is a need in the art to prepare active pharmaceutical ingredients, such as zofenopril or its calcium salt with lesser particle size distribution and high surface area to obtain formulations with greater bioavailability.

Zofenopril calcium of defined particle size may be produced by precipitation from appropriate solvents. Particle size may be adjusted by customary methods such as cooling, pH adjustment, pouring a concentrated solution into an anti-solvent and/or by co-precipitation so as to obtain a precipitate with the appropriate particle size distribution.

Further, zofenopril calcium of defined particle size may be produced by known methods of particle size reduction starting with crystals, powder aggregates and course powder of either crystalline or amorphous zofenopril calcium. The principal operations of conventional size reduction are milling of a feedstock material and sorting of the milled material by size. A fluid energy mill, or micronizer, is an especially preferred type of mill for its ability to produce particles of small size in a narrow size distribution.

The present invention provides crystal particles of zofenopril calcium obtained by the processes herein described, having a specific surface area of about 5 m²/g to about 25 m²/g, as measured by B.E.T. (Brunauer-Emmett-Teller), preferably from about 10 m²/g to about 15 m²/gm, with a mean particle size of about 5 μm to about 50 μm, preferably of about 10 μm to about 20 μm.

The present invention provides crystal particles of zofenopril calcium obtained by the processes herein described having the following characteristics:

Specific surface area of about 14.13 m²/gm, as measured by B. E.T. (Brunauer-Emmett-Teller)
Bulk density: 0.201% w/w; Tap density: 0.282% w/w; Loss on drying of about 0.2215% w/w by T.G.A;

Crystal particles are condensation Aggregate shape, as observed by SEM (Scanning electron microscope), which is substantially as depicted in FIG. 5.

Mean particle size distribution d (0.5): 12.088 μm; d (0.1): 3.902 μm; d (0.9): 33.858 μm. As used herein, the term “μm” refers to “micrometer” which is 1×10^{˜6} meter.

As used herein, “crystalline particles” means any combination of single crystals, aggregates and agglomerates. As used herein “Particle Size Distribution (P.S.D.)” means the cumulative volume size distribution of equivalent spherical diameters as determined by laser diffraction at 1 bar dispersive pressure in a SympatecHelos equipment. “Mean particle size distribution, i.e., d (0.5)” correspondingly, means the median of said particle size distribution.

Specific surface area is defined in units of square meters per gram (m²/g). It is usually measured by nitrogen absorption analysis. In this analysis, nitrogen is absorbed on the surface of the substance. The amount of the absorbed nitrogen (as measured during the absorption or the subsequent desorption process) is related to the surface area via a formula known as the B. ET. formula. BET Surface Area Analyser Model SAA-2000, specifically designed for BET_Surface Area Analysis.

The Specific Surface Area is expressed in meters square per gram of a sample. It is a measure of area covered by Nitrogen gas adsorbed in a mono-layer form.

Crystal particle shapes of zofenopril calcium are analysed by powder sample is spread on the stub and coated with gold ions and observed under scanning electron microscope.

In a still yet further embodiment, the present invention provides zofenopril calcium having a mean particle size less than about 15 μm.

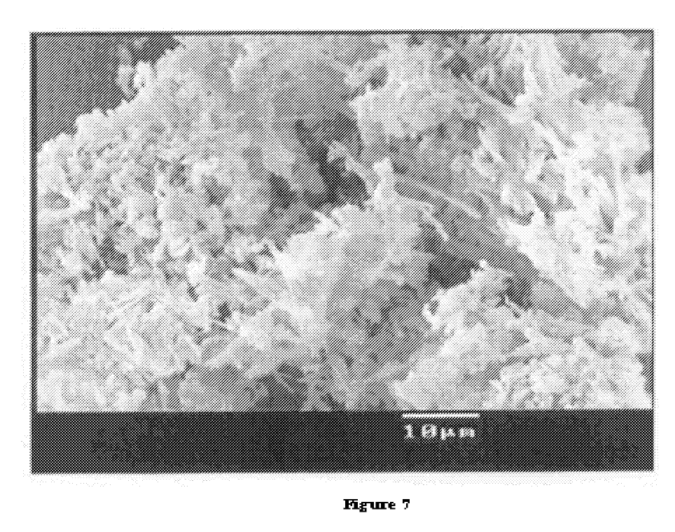

In another embodiment, The present invention provides zofenopril crystal calcium particles have a specific surface area from about 10 m²/g to about 15 m²/g, as measured by B.E.T. (Brunauer-Emmett-Teller) and Aggregate shape as observed by SEM, which is substantially in accordance with FIG. 7.

In yet another embodiment, the present invention provides zofenopril calcium in a polymorphic form mixture comprising Form A and Form; wherein Form B is >20% w/w; specifically Form is in the range of about 20-30%, or Form B is in the range of about 30-40% or Form B is in the range of about 40-50%; or Form B is in the range of about 50-60% or Form B is in the range of about 60-70% or Form B is in the range of 70-80% or Form B is in the range of 80-90% or Form B is >90% wt./wt.

In yet another embodiment, the present invention provides a pharmaceutical composition comprising zofenopril or its pharmaceutically acceptable salts obtained by the processes described herein and at least a pharmaceutically acceptable carrier.

Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes. Oral dosage forms include, but are not limited to, tablets, pills, capsules, troches, sachets, suspensions, powders, lozenges, elixirs and the like. The zofenopril or its pharmaceutically acceptable salts obtained by the process disclosed herein also may be administered as suppositories, ophthalmic ointments and suspensions, and parenteral suspensions, which are administered by other routes. The most preferred route of administration of the zofenopril or its pharmaceutically acceptable salts is oral. The dosage forms may contain the zofenopril or its pharmaceutically acceptable salts as part of a composition. The pharmaceutical compositions may further contain one or more pharmaceutically acceptable excipients. Capsule dosages will contain the zofenopril or its pharmaceutically acceptable salts which may be coated with gelatin. Tablets and powders may also be coated with an enteric coating. The enteric-coated powder forms may have coatings comprising phthalic acid cellulose acetate, hydroxypropylmethyl cellulose phthalate, polyvinyl alcohol phthalate, carboxymethylethylcellulose, a copolymer of styrene and maleic acid, a copolymer of methacrylic acid and methyl methacrylate, and like materials, and if desired, they may be employed with suitable plasticizers and/or extending agents. A coated tablet may have a coating on the surface of the tablet or may be a tablet comprising a powder or granules with an enteric-coating. Tableting compositions may have few or many components depending upon the tableting method used, the release rate desired and other factors. For example, the compositions of the present invention may contain diluents such as cellulose-derived materials like powdered cellulose, microcrystalline cellulose, microfine cellulose, methyl cellulose, ethyl cellulose, hydroxyethylcellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art.

The process for the preparation of zofenopril or its pharmaceutically acceptable salt of the present invention is simple, eco-friendly and easily scaleable.

The following examples are provided to enable one skilled in the art to practice the invention and are merely illustrative of the invention. The examples should not be read as limiting the scope of the invention as defined in the claims, appended herewith.

### EXAMPLES

### Example 1

### Preparation of Zofenopril Tertiary Butyl Amine

250 ml of toluene, 25.0 g (0.096 moles) of 4-phenylthio L-proline hydrochloride and 18.66 g. of triethylamine were charged into a clean and dry 4 neck round bottom flask (RBF) followed by stirring for about 1 hour at about 25° C. to about 30° C. 22.66 g. (0.101 moles) of S-(−) benzoylthio-2-methyl-propionic acid, 13.64 g. (0.101 moles) of 1-hydroxybenzotriazole (HOBT) and 22.79 g. (0.111 moles) dicyclohexylcarbodiimide (DCC) was added at regular intervals of time about every 5-10 mins. The resultant reaction mass was stirred at about 30-35° C. for about 2-3 hours. The reaction progress was monitored by thin layer chromatography (TLC). After completion of the reaction, the reaction mass was cooled to about 5-10° C. followed by stirring for about 2 hrs. The solid (dicyclohexylurea) was filtered on celite and the celite bed was washed with 50 ml of cold toluene. The clear filtrate was taken in a clean and dry 500 ml 4 neck RBF and 100 ml water was charged. The pH of the reaction solution was adjusted to about 9-9.5 with 10% sodium carbonate solution followed by stirring for about 30 min. The aqueous layer was separated and the toluene layer was extracted with 2×100 ml water. Both aqueous layers were combined and 250 ml of methylene dichloride was charged to it. The pH of the above reaction mass was adjusted to below 2 with hydrochloric acid. The reaction mass was filtered and the filtrate was collected into a clean and dry RBF. The organic and aqueous layers were separated and the aqueous layer was extracted with 2×100 ml of methylene dichloride. Both the organic layers were combined and dried over anhydrous sodium sulphate. The organic layer was distilled completely at about 35° C. under vacuum. The oily mass was degassed for 1 hr under high vacuum.

weight of degassed oily mass=47 g; MASS (M−H)=428 (Zofenopril free acid).

225 ml Acetonitrile was added to the oily mass and the reaction mass was stirred to obtain a clear solution at about 25-30° C. 7.96 g (0.109 mol) of tert-butylamine was charged at 25-30° C. The resultant reaction mass was stirred for about 1 hr at about 25-30° C. The reaction mass was heated to reflux at about 80-85° C. for about 90 min. The reaction mass was slowly cooled to about 25-30° C. The reaction mass was cooled to about 0-5° C. and stirred for about 1 hr. The solid separated was filtered and the solid was washed with 150 ml acetonitrile and running wash with 50 ml acetonitrile. The solid obtained was dried at about 40-45° C. under vacuum to afford 42.5 g of the title compound as white crystalline powder.

### Example 2

### Preparation of Zofenopril Freeacid in Solid Form from Zofenopril Tertiary Butyl Amine Salt

10 g zofenopril tert-butylamine salt, 150 ml methylene chloride and 100 ml of water were charged into a clean and dry 4 neck RBF. The pH was adjusted to about 4.0-5.0 with hydrochloric acid. The reaction mass was stirred for about 30 min. Organic and aqueous layers were separated and the aqueous layer was extracted with 2×100 ml of methylene chloride. Total methylene chloride layer was combined and treated with sodium sulphate. The methylene chloride was distilled completely and degassed. Foamy solid mass was obtained in flask. 40 ml of petroleum ether was added to the foamy solid mass. The mass was stirred at about 5-10° C. for about 30 minutes to about 45 minutes under nitrogen atmosphere. The solid separated was filtered under nitrogen, then suck dried under nitrogen until the last droplet of mother liquor. The material was unloaded immediately under nitrogen, packed under nitrogen and kept at about 2-8° C. yield: 8 grams.

### Example 3

### Preparation of Zofenopril Dicyclohexyl Amine Salt

630 ml of acetonitrile was added to the 105 g (0.244 moles) of zofenopril oily mass and the reaction mass was stirred to obtain clear solution at about 25-30° C. 44.3 g (0.244 mole) of dicyclohexylamine was added slowly over about 2-3 hours at about 25-30° C. The resultant reaction mass was stirred for about 1 hr at about 25-30° C. The reaction mass was slowly cooled to about 0-5° C. and was stirred for about 5-6 hours. The solid separated was filtered and the solid was washed with 150 ml acetonitrile and washed with 2×150 ml of diethylether. The solid obtained was dried at about 40-45° C. under vacuum to afford 130 g of the title compound as white crystalline powder.

Purity by Chiral HPLC: 99.8%

### Example 4

### Preparation of Zofenopril Freeacid in Solid Form from Zofenopril Dicyclohexyl Amine Salt

10 g of zofenopril dicyclohexyl amine salt, 150 ml methylene chloride and 100 ml of water were charged into a clean and dry RBF. The pH was adjusted to about 4.0-5.0 with hydrochloric acid. The reaction mass was stirred for about 30 min. The organic and aqueous layers were separated and the aqueous layer was extracted with 2×100 ml of methylene chloride. Total methylene chloride layer was combined and treated with sodium sulphate. The methylene chloride was distilled completely and degassed. Foamy solid mass was obtained in flask. 40 ml of petroleum ether was added to the foamy solid mass. The solid mass was stirred at about 5-10° C. for about 30-45 min. under nitrogen atmosphere. The product was filtered under nitrogen; then sucked dry under nitrogen until the last droplet of mother liquor. The material was unloaded immediately under nitrogen, packed under nitrogen and kept at about 2-8° C. Yield: 6.5 grams.

### Example 5

### Preparation of Zofenopril Calcium from Zofenopril Tertiary Butyl Amine Salt Using Calcium Acetate

9.0 g of zofenopril tert-butylamine and 90 ml of water were charged into a clean and dry 500 ml 4 neck RBF. Adjusted the pH to about 8.0-8.5 with 20% caustic solution, 20 ml water was distilled out under vacuum for removing the tert-butylamine at 55-60° C. Again 20 ml demineralized (DM) water was added and distilled out the 20 ml of water from the reaction mass at about 55-60° C. Again 20 ml DM water was added in the reaction mass. The reaction mass was heated to about 55-60° C. The aqueous solution of calcium acetate in equimolar ratio was charged to the reaction mass at about 55-60° C. The mass was stirred at about 55-60° C. for about 10-12 hrs. The reaction mass was cooled to about 40-42° C. and the solid separated was filtered and washed with 45 ml warm water. The product was sucked dried and further dried at about 50-55° C. under vacuum to afford 7.5 g of the title compound.
Purity by chiral HPLC: 99.86 area % with individual impurity not more than (NMT) 0.1 area % by HPLC and total impurities NMT 0.41 area % by HPLC.
Particle size distribution: d 10: 3.902μ; d 50: 12.088μ; d90: 33.858μ.
Bulk density (Tapped): 0.282 gm/ml; (untapped): 0.201 gm/ml
Specific surface area by B.E.T: 14.13 m²/g.
Residual solvents (OVI): Acetonitrile: 25 ppm; Ethyl acetate, petroleum ether, methanol, dichloromethane, isopropyl alcohol, ethanol, toluene are below detection limit.

### Example 6

### Preparation of Zofenopril Calcium from Zofenopril Tertiary Butyl Amine Salt

9.0 g of zofenopril tertiary butylamine and 90 ml water were charged into a clean and dry 500 ml 4 neck RBF. The pH was adjusted to about 8.0-8.5 with 20% caustic solution, 20 ml water was distilled out under vacuum for the removal of the tertiary butylamine at about 55-60° C. and 20 ml DM water was charged and again distilled out the 20 ml of water from the reaction mass at about 55-60° C. Again 20 ml DM water was charged in the reaction mass. Reaction mass was heated to about 55-60° C. The aqueous solution of calcium chloride dihydrate in equimolar ratio was charged to the reaction mass at about 55-60° C. The resultant reaction mass was stirred at about 55-60° C. for about 10-12 hrs. The reaction mass was cooled to about 40-42° C. The solid separated was filtered and washed with warm water till the chloride ion content is negligible. The solid obtained was dried at about 50-55° C. under vacuum to afford 7.5 grams of the title compound.

### Example 7

### Preparation of Zofenopril Calcium from Zofenopril Solid

5 g of zofenopril and 40 ml water were charged into a clean and dry 4 RBF and the pH was adjusted to about 8.0-8.5 with 20% caustic solution The resultant reaction mass was heated to about 40-45° C. Aqueous solution of calcium chloride dihydrate was prepared (dissolved 1.2 g of calcium chloride dihydrate in 4V water), where 20% volume of the above solution of calcium chloride dihydrate was slowly added or until haziness was observed in the reaction mass at about 40-45° C. The mass was stirred at about 40-45° C. for about 25-30 mins. The resultant reaction mass was heated to about 55-58° C. and stirred for about 4-6 hrs. The reaction mass was cooled to about 35-40° C. The solid separated was filtered and solid was washed with 20 ml of water. The solid obtained was dried at about 50-55° C. under vacuum to afford 4 grams of the title compound.

### Example 8

### Preparation of Zofenopril Calcium from Zofenopril Solid

5 g of zofenopril and 40 ml water were charged into a clean and dry 4-neck RBF and adjusted the pH to about 8.0-8.5 with 20% caustic solution. The reaction mass was heated to about 40-45° C. Aqueous solution of calcium acetate was prepared (dissolved 1.3 g of calcium acetate in 4V water). 20% volume of the above solution of calcium acetate was slowly added or till haziness was observed in the reaction mass at about 40-45° C. The mass was stirred for about 25-30 mins at about 40-45° C. The reaction mass was heated to about 55-58° C. The resultant reaction mass was stirred at about 55-58° C. for about 4-6 hrs. The reaction mass was cooled to about 35-40° C. for about 1 hour. The solid separated was filtered and the solid was washed with 20 ml of water. The solid obtained was dried at about 50-55° C. under vacuum to afford 4 grams of the title compound.

Purity by chiral HPLC: 99.86 area % with individual impurity not more than (NMT) 0.1 area % by HPLC and total impurities NMT 0.41 area % by HPLC.

1. Zofenopril in solid form.

2. The compound of claim 1, in crystalline form.

3. The compound of claim 1, in amorphous form.

4. A process for the preparation of zofenopril of claim 1 comprising
a) providing a solution of zofenopril salt in a mixture of water and organic solvent/s;
b) adding an acid to the solution; and
c) recovering the desired solid form of zofenopril.

5. The process of claim 4, wherein the organic solvent is selected from alcohols, ketones, nitriles, esters, aprotic polar solvents and mixtures thereof.

6. The process of claim 5, wherein the solvent is selected from methanol, ethanol, isopropanol, acetone, acetonitrile, ethyl acetate, N,N-dimethylformamide, dimethylsulfoxide or mixtures thereof.

7. The process of claim 4, wherein the pH of the solution after the addition of the acid is between 1 and 4.

8. The process of claim 4, wherein the zofenopril salt is selected from base salts, alkali metal salts, alkaline earth metal salts, salts with organic bases.

9.-27. (canceled)
